(19) United States
(12) Patent Application Publication
(10) Pub. No.: US 2008/0131867 A1
(43) Pub. Date: Jun. 5, 2008

(54) Method for Determining the Effectiveness of a Treatment for Preeclampsia

(76) Inventor: Hamutal Meiri, Tel Aviv (IL)

Correspondence Address: NATH & ASSOCIATES, 112 South West Street, Alexandria, VA, 22314, US

(21) Appl. No.: 11/885,276

(22) PCT Filed: Feb. 1, 2007
(86) PCT No.: PCT/IL07/00131

§ 371 Date: Aug. 29, 2007

Related U.S. Application Data
(60) Provisional application No. 60/764,344, filed on Feb. 2, 2006.

(51) Int. Cl. A01N 1/02; G01N 33/68; G01N 33/50

(52) U.S. Cl. 435/1.3; 436/86; 435/29

(57) ABSTRACT

A method for determining the effectiveness of a treatment for preeclampsia of a pregnant woman at risk for preeclampsia, the method comprising: (a) determining a first concentration of placental protein 13 (PP13) in a bodily substance of the woman obtained prior to the treatment; (b) determining a second concentration of PP13 in a bodily substance of the woman obtained after initiation of the treatment; and (c) comparing the first and second concentrations to a corresponding normal level of PP13 and, based on the comparison, determining the effectiveness of the treatment. Diagnostic kits for practicing the method are also disclosed.

### FIELD OF THE INVENTION

This invention relates to a method for tailoring medications to treat or prevent preeclampsia, and for monitoring their effectiveness.

### BACKGROUND OF THE INVENTION

The pregnancy disorder known as preeclampsia (PE) is a complication of pregnancy occurring in 5-7% of all pregnant women and it is the second most frequent cause of maternal death during pregnancy (18% of maternal mortality associated with pregnancy in the United States). Preeclampsia is defined as a new onset hypertension developed after 20 weeks of gestation in previously normotensive women. The World Congress of Hypertension in Pregnancy has provided the following definition for diagnosing preeclampsia: a new onset hypertension developed after 20 weeks of gestation of ≧90/140 mm Hg (systolic/diastolic, at least one) measured on two occasions, 4-6 hours apart (and in some cases 4-72 hours apart), coupled to the appearance of protein in the urine corresponding to 300 mg/DL in 24 hours collection or 2+ by dipstick measurement, in women who previously had traces or no protein in the urine. Severe preeclampsia is defined as preeclampsia in which the hypertension has reached 160/110 mm Hg (systolic/diastolic, at least one) coupled to proteinuria of ≧3+ in dipstick or >3 gr/dl in 24 hr. Eclampsia is an emergency situation in which severe preeclampsia is exacerbated into convulsion, stroke and coma that endanger the life of the mother. To avoid such an emergency situation, the woman is delivered to remove the baby and the placenta which cause these effects. HELLP is a severe form of preeclampsia where the major side effects are hemolysis, elevated liver enzyme and low platelets.

Although the proportion of preeclampsia is higher in developing countries, numbers in the U.S. remain high (5-7%). Fifty percent of all PE pregnancies are delivered via Cesarean section as compared with only 15-18% of pregnancies in the entire population. Recovery from a Cesarean section delivery lengthens the recovery time. Not only is the procedure more complicated, but it is also more expensive than vaginal delivery. Women who experience PE disorders during pregnancy have a 9 times higher risk of consequently developing cardiovascular diseases and their life expectancy is significantly lower.

Early-preeclampsia is a severe form of preeclampsia which develops early and necessitates delivery before 37 weeks of gestation (before term). Severe and Early-onset PE are major hazards for both mother and fetus. According to the NICHD, early PE accounts for 20-25% of all cases of PE, which means that 1-2 out of 100 pregnant women will be affected by this complication, and the baby is delivered extremely early, after experiencing a stressful pre-delivery period. The earlier the delivery occurs, the more severe are the complications of the baby, due to its low birth weight and incomplete internal organ maturation, the complications including blindness, motor and cognitive disorders and life-long medical disabilities. Babies born prematurely due to preeclampsia are at increased risk to later develop hypertension, cardiovascular diseases and diabetes. The early preeclampsia cases delivered before 34 gestational weeks (GW) are the most severe ones, responsible for most mortality cases, and the babies born, if they survive, need on average 6-8 weeks in neonatal intensive care units. According to the NICHD this is the group for whom early detection is most essential for its life saving capability and prevention of prematurity. The only current practice to treat preeclampsia is to deliver the mother, and when such a delivery takes place prematurely, a variety of impairments of the newborn baby appear due to lower birth weight, motor and cognitive disabilities, and, in very severe cases, in-partum or after partum death. Many studies have been carried out to identify the risk of developing preeclampsia early.

The early detection of risk provides two major advantages:

1) It enables to manage the risk by close surveillance of the woman. Effective increased surveillance for women at high risk is in compliance with ACOG guidelines, and the increased surveillance in high-risk cases significantly improves outcome. Pregnancy Management Programs were shown to save costs due to close surveillance coupled with education and awareness programs given to the participating pregnant women. Among benefits are a drop of births due to early PE (<34 weeks) only 0.6% of all deliveries compared with the national average of 1.96%, premature delivery reduction to 0.9% of all deliveries compared with the national benchmark of 2.3% and low birth weight due to preeclampsia being 1.3% compared with the national average of 2.9%. The close surveillance allows the pregnant woman to reach a tertiary level medical center before birth, an issue of great significance in community clinics and rural-based health service settings. There, close surveillance enables in certain cases to extend pregnancy duration so as to reduce the severity of the consequences to the baby who is under a risk to be delivered pre-maturely. The other benefit is to buy time to administer treatments and drugs such as antenatal corticosteroids that facilitate the maturation of fetal organs.

2) The early detection enables a longer period for developing drug intervention strategies using various putative agents that are considered to work on the placenta to prevent/reduce the risk. Although there is no gold standard for treatment, a number of candidates have shown promise, including low dose aspirin, low molecular weight heparin, anti oxidants such as vitamin C and E and magnesium sulfate, among others. In all of these studies not all women at risk benefited from the therapeutic intervention. While in some cases there are indications that the intervention was initiated too late, in other cases there is no clear evidence to indicate if the medication used wasn't the right one or wasn't used at the right time or dose. Current studies show that it is necessary to tailor drug intervention to each woman from a putative medications list available today (as well as medications that will become available in due course), and to continuously monitor the effectiveness of the treatment.

Among the current leading agents to prevent preeclampsia are:

(1) Low dose salicylic acid (aspirin) to improve the blood flow to the maternal arteries supplying oxygen and nutrients to the placenta; (2) anti-coagulants such as low molecular weight heparin were found effective in preventing trombophilia and its complications that occur in recurrent and severe preeclampsia; (3) Magnesium Sulfate (MgSO₄) that has so far been proven effective only for treating eclampsia. However, its usefullness in treating preeclampsia remains under debate. Nevertheless, MgSO₄ remains the first-line agent in many institutions for treating women with preeclampsia and HELLP (hemolysis, elevated liver enzymes and low platelet count). (4) Anti-oxidants such as vitamins C and E were shown to reduce the prevalence of preeclampsia among high-risk pregnancies. In many of these treatments, side effects such as brain hemorrhages, neuromuscular blockade and difficult resuscitation can develop and cause complications to the mother and the fetus.

Placental Protein 13 (PP13) is a protein of 15-16,000 MW which may be purified from human placental tissue or prepared by recombinant technology as described in U.S. Pat. No. 6,548,306 (Admon, et al), the contents of which are incorporated herein by reference. Purified PP13 was used to develop an assay for the detection of some pregnancy-related disorders such as intrauterine growth restriction (IUGR), preeclampsia and preterm delivery as described in U.S. Pat. No. 5,198,366 (Silberman), the contents of which are incorporated herein by reference. Both a radioimmunoassay (RIA) and an enzyme-linked immunosorbent assay (ELISA) were developed using labeled PP13 and anti PP13 polyclonal antiserum.

Amino acid composition and sequence analysis of PP13 revealed highest homology to the galectin family—a group of proteins with high affinity to sugar residues which is particularly important in bridging cells to the extracellular matrix (and in differentiation) (Than, N. G., et al (1999) Placenta 20:703-710; Than, et al., (2004) Eur. J. Biochem. 271(6):1065-1078). Indeed PP13 was found by immunohistochemistry to be important in placentation.

U.S. Pat. No. 6,790,625, the contents of which are incorporated herein by reference, discloses monoclonal antibodies to PP13 and a solid-phase immunoassay capable of measuring maternal serum PP13 during the early stages of pregnancy.

WO 04/021012, the contents of which are incorporated herein by reference, discloses a diagnostic method for pregnancy complications based on a number of factors, including PP13 level.

### SUMMARY OF THE INVENTION

It is an object of the present invention to provide a simple in-vitro assay that will allow an attending physician to monitor the effectiveness of putative anti-preeclampsia medications administered to a pregnant woman who is at an elevated risk for or suffering from preeclampsia.

It is another object of the present invention to use an in-vitro or ex-vivo system to tailor drug intervention for preventing or treating preeclampsia from a list of putative anti-preeclampsia medications.

In a first aspect of the present invention, there is provided a method for determining the effectiveness of a treatment for preeclampsia of a pregnant woman at risk for preeclampsia, comprising:
(a) determining a first concentration of placental protein 13 (PP13) in a bodily substance of the woman obtained prior to the treatment;
(b) determining a second concentration of PP13 in a bodily substance of the woman obtained after initiation of the treatment; and
(c) comparing the first and second concentrations to a corresponding normal level of PP13 and, based on the comparison, determining the effectiveness of the treatment.

This aspect of the invention may be referred to at times as the direct method.

Optionally the method of the invention may be continued until delivery to follow the effectiveness of the treatment.

In the present specification, the term “preeclampsia” (PE) includes all types of the disease, including mild, severe, early onset, late onset, PE complicated by intrauterine growth restriction (IUGR), and HELLP, unless specifically indicated otherwise.

The term “a treatment for preeclampsia” includes all types of medical treatments used to prevent, reduce the severity of, or therapeutically treat preeclampsia such as hyperoxia and, in particular, treatments using drugs or food additives. Non-limiting examples of drugs for the treatment of preeclampsia include anti-platelet agents such as low dose aspirin, anti-coagulants such as heparins including low molecular weight heparin, anti-oxidants such as vitamins C and E, and magnesium sulfate, as well as novel experimental treatments with growth factors such as vascular epidermal growth factors (VEGF), treatment with CO, etc.

The term “determining the effectiveness of a treatment” may include both comparing the effectiveness of one type of treatment to another type, comparing the same type of treatment under different conditions (oxygen level, temperature, etc), as well as monitoring the effectiveness of a particular treatment over time.

The term “bodily substance of the woman” includes body fluids (serum, amniotic fluid, urine, saliva) and placenta tissue obtained by way of chorionic villous sampling (CVS), amniocentesis, placenta biopsy or using standardized placenta villi.

The term “normal level of PP13” refers to the level of PP13 found in a bodily substance of a normal, healthy pregnant woman who has not developed preeclampsia or is not at risk to develop preeclampsia. It may also refer at times to the level of PP13 released from explants of placenta obtained after delivery, from cultured placenta cells derived from amniocentesis or from placental villi isolated after chorionic villi sampling from a normal, healthy pregnant woman who has not developed preeclampsia or is not at risk to develop preeclampsia.

The level of PP13 can vary as a function of time (gestational weeks), as a function of the genetic and physical characteristics of the woman such as body mass index, maternal age, ethnicity, and parity, and as a function of the identity of the bodily substance measured. Therefore, when comparing a measured PP13 value from a patient to the normal level of PP13, these parameters should be taken into account. At times, the measured PP13 value will be normalized in order to compare it to the corresponding normal level of PP13.

A woman at high-risk to develop preeclampsia may be determined by: 1) risk factors (such as preeclampsia in previous pregnancy or family history); 2) impaired blood flow to the maternal uterine arteries as assessed by higher pulsatility index measured by Doppler ultrasound; 3) abnormal level of various serum markers such as PP13 in the pregnant woman's body fluids as disclosed in the aforementioned patents and patent applications.

In one embodiment, the first concentration of PP13 is selected from the group consisting of: (a) a predetermined range of median PP13 concentrations for the bodily substance in a plurality of untreated pregnant women at a similar risk for preeclampsia; or (b) a measured PP13 concentration of the bodily substance of the pregnant woman prior to receiving the treatment.

The measurement of changes in PP13 in the bodily substances in accordance with the method of the invention is used to determine if the woman's risk persists, is reduced or is elevated in comparison to her initial risk, and in comparison to the typical values of PP13 in the bodily substances of a plurality of normal and high risk women at the respective weeks of gestation. The continuous redefinition of the woman's risk to develop preeclampsia is used as a means to assess the effectiveness of putative medication therapy to reduce/remove the risk to develop preeclampsia.

In one embodiment of the invention, the comparison is made between single measurements of the first concentration and the second concentration. In another embodiment, the comparison is made between a first slope calculated from a plurality of the first concentrations measured at two or more succeeding time points during the pregnancy of the woman and a second slope calculated from a plurality of the second concentrations measured at two or more succeeding time points during the pregnancy of the woman. In a preferred embodiment, the plurality of concentrations is determined over a period of 2-3 weeks. In a further embodiment, the plurality of each of the concentrations is compared to a corresponding plurality of normal levels of PP13.

One embodiment of the invention involves placenta tissues. Assays are carried out in tissue cultured for 1-7 days in tissue culture medium following a treatment given in-vivo. Another embodiment of the invention involves exposing the tissue to putative medications and selecting the most effective one. Although this saves a woman from exposure to an un-necessary medication, she is exposed to an interventional procedure of risk. Thus, the use of placental tissue in the method of the invention is usually suitable only to those undergoing in any event an interventional sampling of placenta tissue. Occasionally, women who have repeated history of preeclampsia and are considered at very high risk for preeclampsia may be offered this approach as well.

Thus, the invention also includes a method for determining the relative effectiveness of two or more different treatments for preeclampsia, the method comprising:
(a) determining a first concentration of PP13 in a placental tissue explant of the woman obtained prior to the treatment;
(b) contacting the explant with a first treatment;
(c) determining a second concentration of PP13 in the explant after the treatment;
(d) comparing the first and second concentrations to a corresponding normal level of PP13 and, based on the comparison, determining the effectiveness of the first treatment;
(e) repeating steps (a) to (d) with one or more additional treatments; and
(f) comparing the relative effectiveness of the two or more different treatments.

In one embodiment, the effectiveness of the treatment is determined in step (d), as follows:
(a) if there is no significant difference between the first and second concentrations, the treatment is determined as ineffective;
(b) if the difference between the second concentration and the normal level of PP13 is significantly less than the difference between the first concentration and the normal level, the treatment is determined as effective;
(c) if the difference between the second concentration and the normal level of PP13 is significantly greater than the difference between the first concentration and the normal level, the treatment is damaging.

In a preferred embodiment, the second concentration is measured within 1-4 days after the placenta explant is contacted with the treatment

A second aspect of the invention relates to a method for determining the relative effectiveness of two or more different treatments for preeclampsia of a pregnant woman at risk for preeclampsia, comprising:
(a) providing a plurality of placental tissue explants standardized for release of PP13 in response to various preeclampsia treatments;
(b) contacting a bodily substance of the woman with a first placental tissue explant;
(c) contacting the explant with a first treatment and determining the concentration of PP13 of the explant after the first treatment;
(d) repeating steps (b) and (c) with one or more additional explants and treatments; and
(e) determining the difference between the concentrations of PP13 after the treatments and a corresponding normal level of PP13, the treatment resulting in the smallest difference being the most effective.

This aspect of the invention may be referred to at times as the indirect method.

In accordance with this aspect of the invention, placental tissue explants that have been standardized for their response in the presence of various medications may be used to assess the displacement/augmentation/blockade effect of various pregnant woman bodily substances on the effect of the medications on the standard tissue explants. In a preferred embodiment, the standardized placenta explants are cryo-preserved before use.

Also included in this aspect of the invention is a diagnostic kit for carrying out the method of the invention comprising (a) a set of anti-preeclampsia drugs; and (b) a set of standardized placenta explants. In one embodiment, the kit further comprises computer software providing a calculation model to determine the effectiveness of the drugs based on the measured PP13 values. Also included in the invention are kits to measure PP13 adjusted to be used in the method of the invention.

As illustrated in FIG. 1, placenta explants (either standard ones or ones obtained from the woman) may be grown in culture conditioned medium for 48 hours or longer in the presence of various putative anti-preeclampsia medications. In accordance with the first aspect of the invention in the case of a woman's own tissue, a comparison is made of PP13 released in conditioning medium with/without medication. In accordance with the second aspect of the invention in the case of a standardized tissue from another source, the tissue is apportioned: one portion is used to measure the impact of drugs in the absence of the woman's bodily substance whereas the other portion is tested in their presence. In the case of a woman at risk for preeclampsia, her bodily substance influences PP13 release as compared to explants that were not exposed to the patient's bodily substance. The comparison enables one to assess the value of the treatment with the medication in view of molecules included in the bodily substance of the patient. In all cases, the culture supernatant is collected, centrifuged, and assayed, for example in an ELISA in-vitro assay, to determine the level of PP13 released to the medium from the explant. The level of released PP13, adjusted to the protein level or tissue weight and culture viability and compared to a control and standard conditions, is used to assess the impact of the medication.

The comparative analysis of PP13 release enables one to identify which medications are capable of bringing PP13 release back to its normal level as in unaffected patients, and such effect is taken as an indication of the curative drug effect. Based on the above, it would be possible to select one of a plurality of various candidates of medications/combinations of medications of the currently existing protocols that would have the highest likelihood of being effective.

According to the current invention, a woman of established risk can be followed throughout her pregnancy, and her risk is assessed in order to verify how various treatments affect her risk. PP13 from bodily substances is used in this invention to evaluate the woman's risk. Accordingly, the analysis is not based on a plurality of cases and controls but is adjusted to individuals and their specific responses. In this way it is possible to assess therapeutic benefits/effects and to differentiate between them for individuals, thereby minimizing the trial and error process.

The term “abnormal level of PP13” may be defined in relation to the gestational age according to the three major pregnancy periods:
(a) Gestational weeks 6-13: high-risk is associated with low PP13 value (PP13 at the lower population quartile or, in one embodiment, PP13 multiple of the Median (MoM) below approximately 0.45).
(b) Gestational weeks 14-25: a steep increase of PP13 values as compared to the values in the previous period, or as compared to the values of a normal risk group of pregnant women. In one embodiment, the increase in PP13 values has an average slope of 7.
(c) Gestational week 26-to-delivery: PP13 value above normal (in one embodiment with a MoM of >1.5 of the highest quartile than or higher than values calculated from a plurality of normal pregnant women).

Accordingly, a drug benefit is determined in relation to its ability to bring the PP13 level of the respective pregnancy period back to the normal level. Thus, in the first trimester a drug benefit is determined according to its ability to elevate PP13 level/release to the normal (higher) level. In the second trimester it is assessed by a drug ability to reduce the steepness of the slope of change in PP13 release from high (e.g. 7) to normal (e.g. <3). In the third trimester the benefit is judged according to the ability to reduce the PP13 level back to normal.

In the following examples it is demonstrated how measuring PP13 could benefit in the assessment of elevated risk to develop preeclampsia and how one might use the PP13 risk assessment tool to identify a beneficial drug or to tailor drug intervention.

### BRIEF DESCRIPTION OF THE DRAWINGS

In order to understand the invention and to see how it may be carried out in practice, certain embodiments will now be described, by way of non-limiting example only, with reference to the accompanying drawings, in which:

FIG. 1 shows the procedure for obtaining placenta explants from the chorionic villi or the apical membrane of the placenta syncytiotrophoblast. From left to right, the explants are shown at transfer (6 hours) and after 36 hr in culture. Cultures are grown in conditioned medium. After 48 hr in culture, the supernatant is collected, centrifuged and the pellet is diluted in PBS and checked for the concentration of PP13 by sandwich ELISA using PP13 standards to calibrate the reaction OD to PP13 concentration. The PP13 level may be normalized to the gestational age according to the linear regression of the total points and to the protein level of the explant;

FIG. 2 is a graph illustrating longitudinal assessment over time of PP13 level (pg/ml) measured every 2-4 weeks in blood samples of 52 women who delivered at term a normal baby, and 5 women who developed severe preeclampsia around term. Delivery time is indicated by vertical lines. Each curve represents the anticipated serum level of PP13 for a plurality of normal or diseased women;

FIG. 3 is a bar graph which shows division of first trimester PP13 levels (in pg/ml) of 250 normal women into four quartiles, and the subsequent designation of 50 women at high risk for preeclampsia to the various quartiles. According to this method, a shift of the woman from a lower quartile to a higher one is an indication of the drug benefit;

FIG. 4 is a bar graph which shows the PP13 values over three gestational periods of 1179 normal pregnant women, 20 women at risk for preeclampsia, 40 normal women who were treated with vitamin E, and 19 women at risk treated with vitamin E.;

FIG. 5 is a bar graph showing the rate of false positive results (i.e. cases where the results based on PP13 measurement indicate that the woman will develop preeclampsia, but in actuality she didn't) based on measurement of the PP13 serum level during the 1^{st} trimester (PP13 MoM), based on the 2^{nd} trimester assessment of the slope between two time points (PP13 slope), based on a combination of the two measurements (Combined) and based on a combination of the measurements in women with low 1^{st} trimester values (Contingent);

FIG. 6 is a graph showing PP13 (pg/nl) release from trophoblasts in culture over time (days) obtained from women who have a normal pregnancy or are at risk for preeclampsia, with or without treatment with vitamin C and magnesium;

FIGS. 7-11 are bar graphs which demonstrate median results ±95% confidence intervals of PP13 levels (pg/ml) obtained with different anti-preeclampsia means (such as 20% oxygen—hyperoxia) and drugs in standardized placental explants obtained from women at risk (12 women at risk to develop preeclampsia [grey columns] and 3 women at risk to develop HELLP [dotted columns]) and 16 normal controls [empty columns] *=p<0.01; **=p<0.001. The tissue of each woman was divided into 16 different portions, exposed each to diversified conditions and cultured for 48 hours;

FIG. 7 illustrates the effect of 6% and 20% oxygen on PP13 release;

FIG. 8 illustrates the effect of 6% and 20% oxygen, and 0.7 and 1.4 mM Mg on PP13 release;

FIG. 9 illustrate the effect of 6% (FIG. 9A) and 20% oxygen (FIG. 9B), 0.7 and 1.4 mM Mg, and vitamins C and E on PP13 release. M—conditioned medium with 0.7 mM Mg; MC—M+vitamin C; ME—M+vitamin E; MM—conditioned medium with 1.4 mM Mg; MMC—MM+vitamin C; MME—MM+vitamin E. Best candidates are indicated by arrows;

FIG. 10 illustrate the effect of 6% (FIG. 10A) and 20% oxygen (FIG. 10B), 0.7 mM Mg, and the anti-coagulents heparin and aprotinin on PP13 release. M—conditioned medium; MH—M+heparin; MA—M+aprotinin; MM—conditioned medium with 0.7 mM Mg; MMH—MM+heparin; MMA—MM+aprotinin. Best candidates are indicated by arrows;

FIG. 11 show further results using the conditions presented in FIG. 10. FIG. 11A—6% oxygen+0.7 mM MgCl₂; FIG. 11B—6% oxygen+1.4 mM MgCl₂; FIG. 11C—20% oxygen+0.7 mM MgCl₂; FIG. 11D—20% oxygen+1.4 mM MgCl₂. M—conditioned medium with 0.7 mM Mg; MH—M+heparin; MA—M+aprotinin; MHA—M+heparin+aprotinin; MM—conditioned medium with 1.4 mM Mg.

### DETAILED DESCRIPTION OF EXEMPLARY EMBODIMENTS

### Methods

Unless otherwise indicated, in all experiments, PP13 was measured (blinded to pregnancy outcome) in maternal venous serum by solid-phase sandwich ELISA assay. The PP13 level was calibrated according to standard curves prepared from calibrated standards of recombinant PP13. Concentrations are given in pg PP13/ml serum.

The various clinical studies were approved by the medical center Internal Review Ethical Committee, and all women enrolled in the study provided informed consent to allow the use of a small volume of their body fluids to determine the level of PP13. Patients were not randomized for any treatment but the decision to treat was based on the standard of medical care in the hospital as best suited to each individual patient. All other details are described for each example alone.

### EXAMPLES

### Methods to Assess the Risk for Preeclampsia by PP13 and to Evaluate its Increase/Decrease

In the context of assessing the risk for preeclampsia long before clinical symptoms appear, the following methods in accordance with the invention may be used to determine the risk based on PP13 testing:
(1) Measuring a woman's PP13 level by Sandwich ELISA with PP13 standards and a pair of PP13 specific monoclonal antibodies as is known, obtaining the data in pg/ml and comparing the level to the normal median ±95% confidence interval. A cutoff is then established at an optimal sensitivity/specificity trade-off to determine a PP13 serum level required to identify women at elevated risk. A significant difference of a woman's value from the anticipated normal median establishes the risk.
(2) Calculating the multiple of the medians (MoM) of the normal plurality after measuring PP13 according to method (1). In this way the median MoM of the normal PP13 is established. This is normalized to gestation week (GW) reference medians of normal PP13 values and further adjusted linearly to maternal weight or body mass index (BMI).
(3) To standardize raw PP13 values within and across laboratories, reference medians for normal outcome at each gestational week are determined by regressing the raw values of at least 40 raw PP13 values per each gestational week over gestational weeks and extracting from the regression line the reference gestational week specific median. MoM is then calculated as follows:

$$MoM = \frac{PP {13}_{ij}}{{Median}_{j}}$$

Where: i=Gestation Week and j=Subject.
The, MoMs are then regressed over BMI categorized into 4 values of the BMI quartiles and adjusted accordingly. PP13 results are thus standardized. The use of this procedure enables the combining/comparing data across laboratories.
(4) The normal plurality PP13 MoM is defined as 1.0. During the first trimester, MoM=0.45 provides a cutoff under which the patient likelihood of developing preeclampsia is at least 4 times higher compared to the normal population with a >80% sensitivity and >85% specificity. Accuracy is defined by why or receiver operational characteristic (ROC) curves where an area under the curve (AUC) of 0.5 provides no prediction, an AUC>0.75 with 95% confidence interval (CI) of [0.65-0.85] provides a fair prediction and an AUC>0.8 with 95% CI [0.7-0.9] provides very good prediction with P<0.05 and above. Women with MoM below 0.45 are considered as being at elevated risk.
The use of MoM is exemplified in Table 1. During the first trimester, in GW 5-10 and 11-15, women with elevated risk to develop preeclampsia have a PP13 MoM of 0.14 and 0.17, respectively, which are significantly below the normal values as indicated by p<0.001.
In the second period of gestational weeks 16-20 and 21-25, the respective MoMs increase from the earlier very low levels to 0.59 and 1.08 corresponding to p<0.05 and 0.39, respectively. The example above shows the benefit of the MoM method in the first trimester and the need to switch to another method based on PP13 change over time for the subsequent period as detailed in (5) below.
(5) Measuring the slope of PP13 change over time by performing two tests of PP13, a few weeks apart, and calculating PP13 Slope=(PP13_{GW2}−PP13_{GW1})/(GW₂−GW₁), where 1 and 2 represent GW of an earlier and a later time point, respectively, between which the slope was measured. Comparing to a slope of a plurality of normal pregnant women which is much lower compared to women of elevated risk for preeclampsia provides an additional risk parameter. A cutoff is then established to identify a PP13 Slope required to identify women at elevated risk at an optimal sensitivity/specificity trade-off.
(6) Estimating the woman's likelihood ratio (LR) to develop the pathology based on a model. In this approach the mode involves estimating the LR for the possibility of a woman to develop preeclampsia given the PP13 level relative to the possibility of a normal outcome. Examination of the MoM values showed that PP13 distribution cannot be fitted to a Gaussian distribution, either un-transformed or after a log transformation. Therefore, logistic regression was performed in order to model LRs, assuming PP13 level determines the risk. The logistic regression provides the odds ratio (OR) for preeclampsia. It may then be computed:

LR_{PP13}=OR_{PP13}/P
where P is the percentage of preeclampsia in the pregnant population.

For any of the methods described above, the data is fit into a statistical model to plot the receiver operating characteristic (ROC) curves to evaluate the cutoffs of the measures that are required to establish the sensitivity and specificity to distinguish between cases (of preeclampsia) and controls. The use of MoMs, Slopes and LRs were found to be independent of the population examined and the lab that performed the testing, whereas PP13 concentration varied according to the above. Thus, the use of the three latter measures provides independent population standards whereas the exact concentration (pg/ml) may vary between population groups and laboratories.

### Longitudinal Monitoring

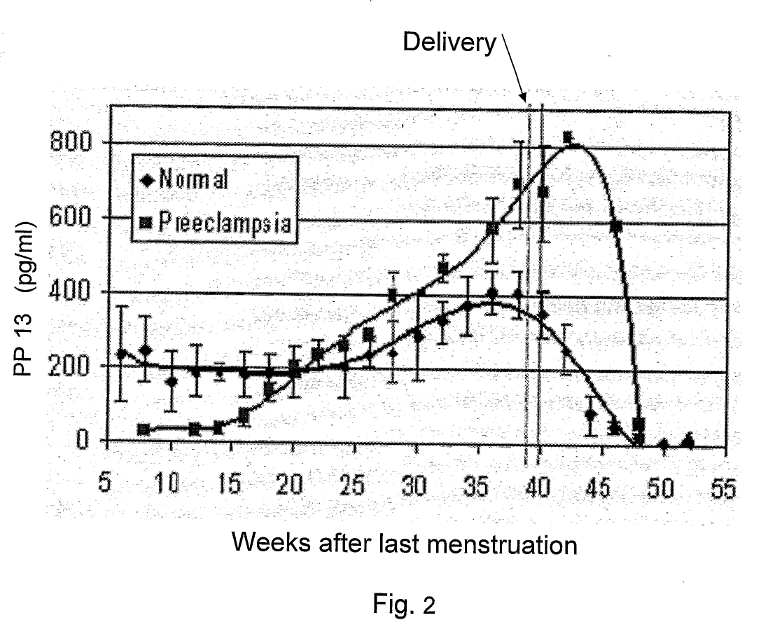

FIG. 2 depicts results of longitudinal monitoring over time of PP13 in serum of normal and preeclamptic pregnant women. Accordingly, one could identify several parameters to differentiate normal unaffected women from preeclamptic ones. For monitoring drug effectiveness, an effective drug should decrease the differences between normal and high risk patients. Table 1 presents the data of FIG. 2 after obtaining the MoM values. Statistical analysis was carried out to compare PP13 MoM normal and preeclamptic values in each corresponding testing period. The results indicate that during the 1^{st} and the 3^{rd} trimesters but not during the 2^{nd} trimester Mom PP13 values are significantly different between preeclamptic and normal women (preeclamptic being very low in the first trimester and very high in the third while normalizing in the second). Furthermore, the table shows how treatment with anti-coagulants as compared to placebo can modify the MoM of un-treated at-risk women (but not of controls).

TABLE 1
PP13 MoM throughout Pregnancy

| | Median MoM (±95% Confidence Interval) | | | |
|---|---|---|---|---|
| | Placebo | | | |
| | | High-Risk for | Anti-Coagulants | |
| | Normal | Preeclampsia | Normal | High-Risk for Preeclampsia |
| GW | Risk (n = 48) | (n = 5) | (n = 5) | (n = 3) |
| 5-10 | 1.00 | 0.14** | 1.00 | 0.17** |
| | (0.16) | (0.03) | (0.16) | (0.05) |
| 11-15 | 1.00 | 0.17** | 1.00 | 0.48* |
| | (0.25) | (0.06) | (0.25) | (0.12) |
| 16-20 | 1.00 | 0.59* | 1.00 | 0.73 |
| | (0.29) | (0.09) | (0.29) | (0.23) |
| 21-25 | 1.00 | 1.08 | 1.00 | 0.91 |
| | (0.18) | (0.03) | (0.18) | (0.23) |
| 26-30 | 1.00 | 1.49* | 1.00 | 1.18 |
| | (0.26) | (0.31) | (0.26) | (0.25) |
| 31-35 | 1.00 | 1.72** | 1.00 | 1.27 |
| | (0.12) | (0.1) | (0.12) | (0.27) |
| 36-40 | 1.00 | 1.76** | 1.00 | 1.21 |
| | (0.14) | (0.17) | (0.14) | (0.21) |
| 40-45 | 1.00 | 1.08 | 1.00 | 1.00 |
| | (0.73) | (0.32) | (0.37) | (0.34) |
| Outcome | All Normal | 4 Severe Preeclampsia | All | 1 Severe preeclampsia, 1 Mild |
| | | 1 Normal | Normal | Preeclampsia 1 Normal |

(*p < 0.05, **p < 0.001)

### 1) First Trimester

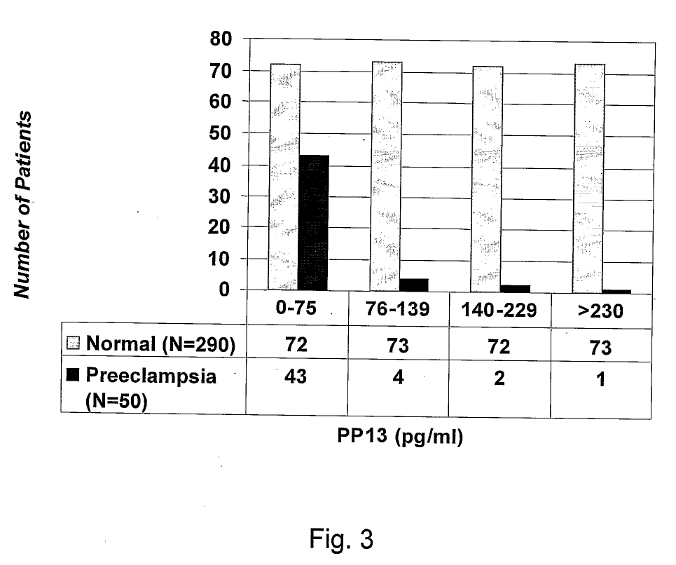

First trimester PP13 could serve as a measure to assess the risk for a later development of preeclampsia. According to FIG. 2 and Table 1, in the first trimester, the majority of the women who will go on to develop preeclampsia have very low levels of PP13 while most women who will have a normal outcome have higher PP13 values. In one study summarized in Table 2 and FIG. 3, the PP13 level was measured at 8 weeks of gestation from 50 cases who went on to develop preeclampsia and in 290 cases with normal outcome. The method of multiples of the gestation-specific median (MoM) was used, and at MoM cutoffs of 0.45, the false positive rate was 10% and the sensitivity 87%. This means that 43 out of 50 preeclampsia cases and 29 out of 290 normal were identified as being at high risk. Thus, while the risk for PP13 in the population is 5%, in the group with PP13 below 0.45, the frequency of preeclampsia was 59% (more than 10 times above the frequency in the population not tested).

Sensitivity and specificity were calculated from the receiver operating characteristic (ROC) analysis based on the indicated MoM cutoffs of 0.45. Sensitivity value is provided in Table 2 in percentile for 10% false positive rate. The odds ratio for developing the pathology was determined by two methods of calculations:

1) modeling, that calculate the Odds ratio as already described above after taking into consideration the prevalence of the pathology in the population, and

2) the quartile assessment procedure, of calculating of the odds ratio for the development of preeclampsia based on the comparison of PP13 in the lowest quartile (25%) versus PP13 in the third quartile (75th percent).

The results of these two methods of calculations are provided in Table 2 and the illustration of the quartile method is provided in FIG. 3. According to FIG. 3, women who had tested in the 8 h gestational week were followed until delivery. 290 had normal delivery. Dividing them into four groups enabled to identify the four quartiles of PP13 with the respective PP13 concentration in each quartile being 0-75 pg/ml, 76-139, 140-229, and above 229. The preeclampsia cases were then assigned to the 4 quartiles according to their PP13 values. FIG. 3 shows that 86% of all women who went on to develop preeclampsia were in the lower quartile, 8% were in the 2^{nd} quartile, 4% in the 3rd quartile and 2% in the 4^{th}.

### Example 1

### Assessment of the Effectiveness of Treatment by Low Dose Aspirin

If in the period specified above a medication is used, it is anticipated that it will bring a woman's PP13 level to the 2nd or 3rd or even the 4th quartile, corresponding to her reduced likelihood of developing preeclampsia. In the example described in Table 2, women with elevated risk to develop preeclampsia were orally treated from GW8 with a dose of 100 mg/kg aspirin (“low dose aspirin”) for either 2 or 3 weeks. It has been suggested that aspirin given early enough could reduce the risk of later development of preeclampsia. Accordingly, women who were treated were anticipated to have lower risk to develop preeclampsia and their outcome should also be improved.

In the study shown in Table 2, of 150 women tested as being at high risk in the 8^{th} week, 50 were not treated, 50 were treated with aspirin for two weeks and 50 were treated for 3 weeks. The results showed that in the untreated group, most women remained in the low PP13 quartile. In the groups treated for 2 or 3 weeks, the numbers of patients in the 2^{nd} and 3^{rd} quartiles were elevated significantly compared to the first quatrile. The calculation of their anticipated risk was reduced accordingly. Delivery outcome corresponded to the risk assessment, where the number of preeclampsia cases was significantly lower in the treated vs. untreated groups.

Accordingly, the frequency shift from 1^{st} to 2^{nd} and 3^{rd} quartile could serve as a measure to assess the reduction in the risk to develop preeclampsia and the effectiveness of a treatment.

TABLE 2
Patients Allocated to PP13 Quartiles.

| Parameter | Untreated | 14 days Aspirin | 21 days aspirin |
|---|---|---|---|
| Sensitivity (%) | 87% (80-94) | 40 (26-56) | 30 (14-46) |
| Frequency of 1^{st} | 43/50 | 20/50 | 14/50 |
| quartile cases | | | |
| Frequency of 2^{nd} | 4/50 | 24/45 | 30/50 |
| quartile cases | | | |
| Frequency of 3^{rd} | 2/50 | 5/50 | 5/50 |
| quartile cases | | | |
| Odds ratio (by | 77.6 | 5.9 | 3.3 |
| modeling) | | | |
| Odds ratio (by | 73.7 | 5.6 | 3.2 |
| quartiles) | | | |
| Outcome | All 50 | 24 Preeclampsia | 18 Preeclampsia |
| | Preeclampsia | 26 Normal | 33 Normal |

Sensitivity in percentile values are shown when the specificity was fixed to 90% (95% CI: 86%-93%), corresponding to having 29/292 cases of false positives (10%).

### Example 2

### Assessment of the Effectiveness of Treatment by Anti-Coagulant Drugs

Women were identified as being at elevated risk and were treated from the 8^{th} week of gestation with anti-coagulants (low molecular weight heparin, aprotinin or others) given daily for 2 weeks. Their PP13 MoM was found to be elevated to 0.48 MoM (GW11-15) (P<0.05), and 0.73 (GW16-20), respectively, with the latter being practically indistinguishable from the normal level (1±0.29, Median normal MoM±95% Confidence Interval). PP13 MoM of women with normal risk was not affected. The corresponding outcome of the treated women was: with no treatment, all 5 women with elevated risk developed severe preeclampsia around term, whereas in the treated group one developed severe preeclampsia, one mild preeclampsia and one was unaffected.

### Example 3

### Assessment of Drug Benefit Using Placental Extract

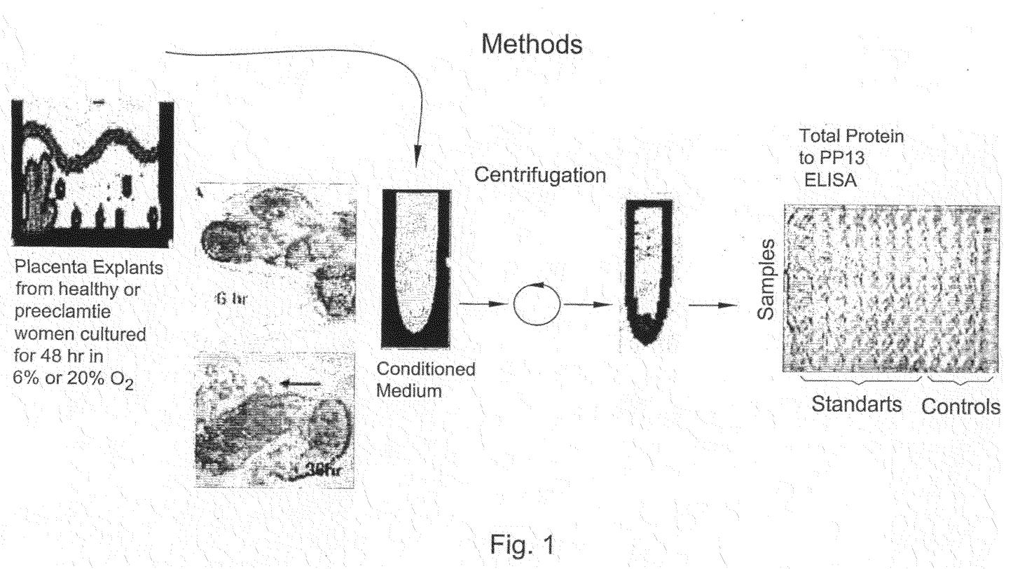

An alternate method of assessing drug benefit is by using placenta villi (cells or explants) obtained during gestation week 9-10 from pregnant women undergoing chorionic villi sampling. The placenta cells/explants were cultured for 48 hr and PP13 was measured in the culture medium by ELISA (in the same manner as described in FIG. 1). The results are shown in Table 3 below.

Table 3 shows that for the 3 cases of preeclampsia (cases #3, 4 and 5), the amount released under 6% oxygen (normoxia) is much lower (3,010, 3,500 and 6,300) as compared to 14,100 and 15,700 in normal women (cases #1 and 2). After 48 hours incubation with the anti-oxidant vitamin C, that has shown promise in treating high-risk women, the level of PP13 release is brought up almost to the normal level in all 3 high risk women, reaching 12,030, 9,230, and 15,790, respectively (i.e. 3-4 times higher). Under 20% oxygen (hyperoxia), PP13 release increased to 5000, 4,300 and 7,900, respectively, due to the oxygen itself (approximately by a factor of 2 as compared to 6% oxygen: 3,010, 3,500 and 6,300 pg/ml), while there is no additional effect of vitamin C.

As can be seen, not all individual women treated this way show the same effect, indicating the potential of the method of the invention to be further used to consider discontinuing the treatment, elevating the drug level or selecting a different treatment for individual patients. Although the use of a placental extract for assessing preeclampsia risk saves a woman from exposure to an un-necessary medication, she is exposed to an interventional procedure of risk. Thus, this is suitable only to those women undergoing an interventional sampling of placenta tissue in any event.

TABLE 3
PP13 Release from Cultured Chorionic villi of GW 9-10

| | | | | PP13 | Pregnancy |
|---|---|---|---|---|---|
| Case # | Risk | Oxygen (%) | Vitamin C | (pg/ml) | outcome |
| 1 | Normal | 6 | No | 14,100 | Normal |
| | | | Yes | 14,300 | |
| | | 20 | No | 14,250 | |
| | | | Yes | 14990 | |
| 2 | Normal | 6 | No | 15,790 | Normal |
| | | | Yes | 15,390 | |
| | | 20 | No | 15,500 | |
| | | | Yes | 15,300 | |
| 3 | High-Risk | 6 | No | 3,010 | Preeclampsia |
| | | | Yes | 12,030 | |
| | | 20 | No | 5,000 | |
| | | | Yes | 5,100 | |
| 4 | High-Risk | 6 | No | 3,500 | Preeclampsia |
| | | | Yes | 9,230 | |
| | | 20 | No | 4,300 | |
| | | | Yes | 4,421 | |
| 5 | High-Risk | 6 | No | 6,300 | Preeclampsia |
| | | | Yes | 15760 | |
| | | 20 | No | 7,900 | |
| | | | Yes | 8,100 | |

2) 1^{st}-to 2^{nd} Trimester Slope

### Example 4

### Assessment of Drug Benefit Using the 1^{st}-to 2^{nd} trimester slope

As already demonstrated in FIG. 2, in normal women PP13 level is only moderately changed between the 1^{st} and the 2^{nd} trimester. The slope may be calculated as follows:

Slope=(PP13_{(2nd trimester)}−PP13_{(1st trimester})/GW_{(2nd trimester)}−GW_{(1st trimester)}).

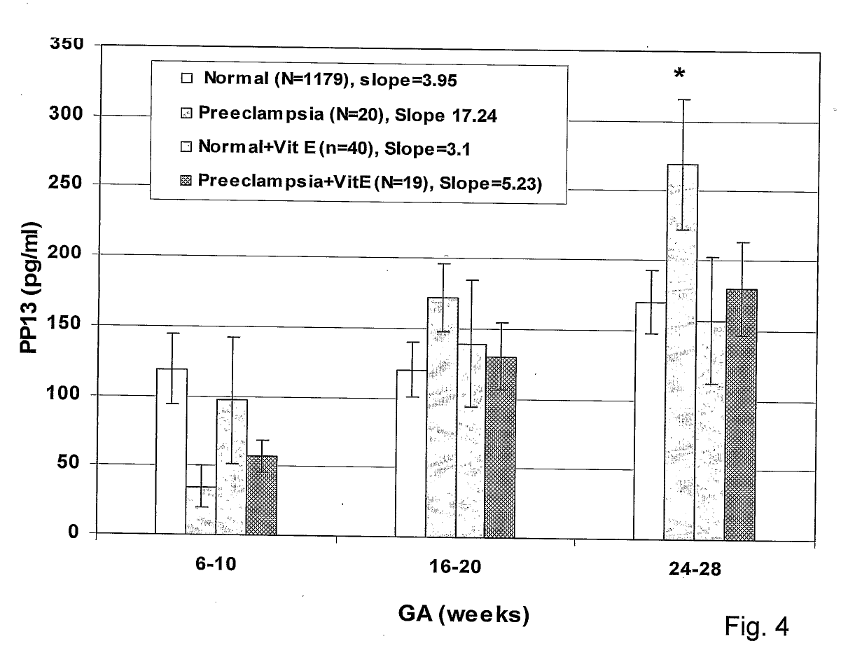

Normal vs. preeclampsia slopes are shown in FIG. 4, and the cutoff between normal and preeclampsia to reach 80% sensitivity is a slope of 3.5. The slope helps to further verify the risk for preeclampsia. The results in FIG. 4 show that in the cases of preeclampsia at 6-10 weeks, the PP13 level is lower than in the normal cases, and early application of vitamin E doubles PP13 release towards the normal level without affecting PP13 release in normal patients. At 16-20 weeks, no significant effect of vitamin E can be demonstrated. At 24-28 weeks, when PP13 release in preeclamptic women is higher than in normal women, vitamin E reduces PP13 release back to the normal level.

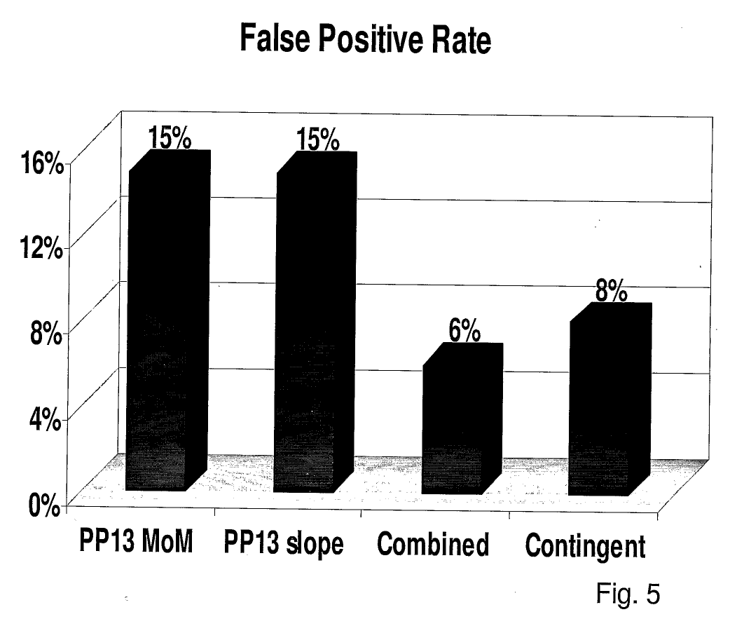

FIG. 5 shows how the level of false positives for a fixed prediction sensitivity is reduced by a combined analysis using both PP13 MoM level in the 1^{st} trimester and the 1^{st}-to-2^{nd} trimester slope. The figure indicates that either first trimester MoM and the slope provide each a 15% false positive rate with 80% sensitivity. Taking the two parameters combined—both first trimester MoM and first-to-second trimester slope by way of combined analysis enabled reducing the false positive rate to 6% without losing sensitivity. In a contingency approach (“Contingent”), only women with low first trimester MoM were tested again and the combined analysis shows that for the same sensitivity, the false positive rate is 8%. Accordingly, it appears that second trimester testing is a must for those identified at risk in the first test.

If after establishing the risk by two tests the women at high risk are treated daily by administering the anti-oxidant vitamin E, the treated women have a lower slope (FIG. 4). From the relatively large confidence level one can see that not all women were affected in the same way. Thus, the approach can be further used to enable one to see how the slope can be reduced by medications and its correlation to the reduced risk to develop the pathology, thereby considering discontinuing the treatment, elevating the drug level or selecting a different treatment for individual patients. The combined and contingent approaches indicate that multiple testing is necessary only for women at risk whereas low risk patients may avoid repeated testing.

### Example 5

### Assessment of Drug Benefit Using Trophoblasts

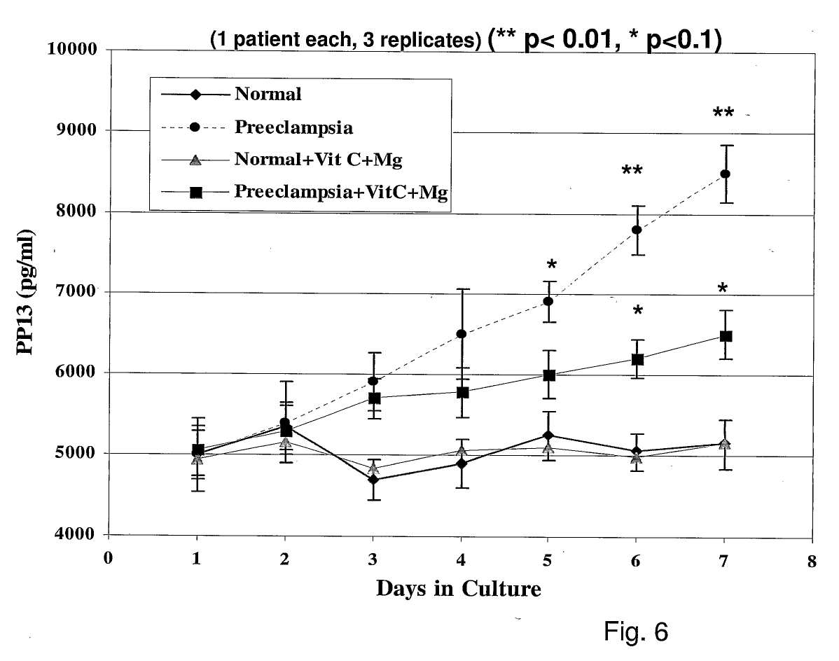

The subject can also be monitored by looking at trophoblasts obtained from amniotic fluid. For example, the trophoblasts may be grown for two weeks under culture conditions with and without a combination of 1.4 mM MgCl₂ and Vitamin C. Trophoblasts in culture from women who went on to develop preeclampsia showed a day to day elevated PP13 release (indicating elevated risk to preeclampsia) as compared to trophoblasts obtained from normal women, whose PP13 release remained practically unchanged (FIG. 6). Culturing the trophoblasts with Mg and Vitamin C had no effect on the normal cultures but prevented the increased PP13 release compared to untreated trophoblasts, indicating a method for determining the treatment effectiveness/ineffectiveness for individual cases. Again, it is anticipated that there will be cases that will not respond, and thus the procedure could be used to “tailor” treatment by continuing, discontinuing or replacing with a different drug.

### 3) Third Trimester

### Example 6

### Assessment of Oxygen Benefit

The following results were obtained with placental (villous) explants from 16 cases of normal women, 12 cases of preeclampsia and 3 cases of HELLP women, which were cultured in DMEM/F12 for 48 hours at 6% or 20% O₂. Conditioned media was collected after culture and tested for total protein and PP13. PP13 release was related to total protein.

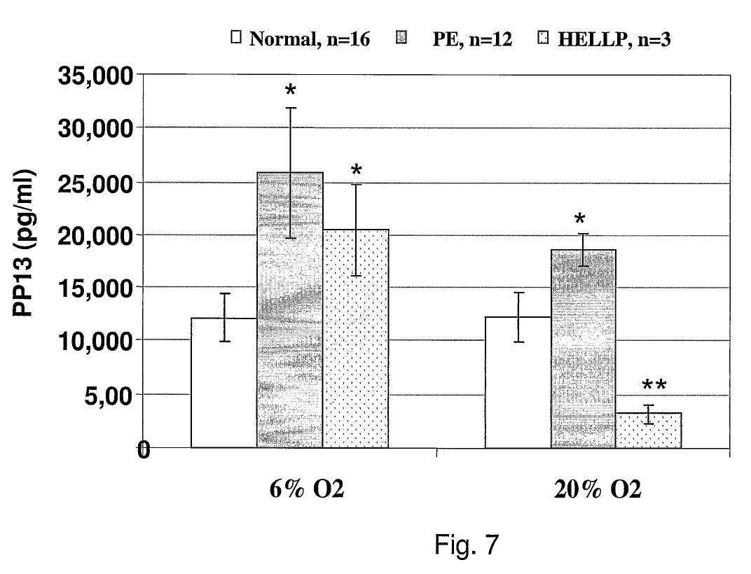

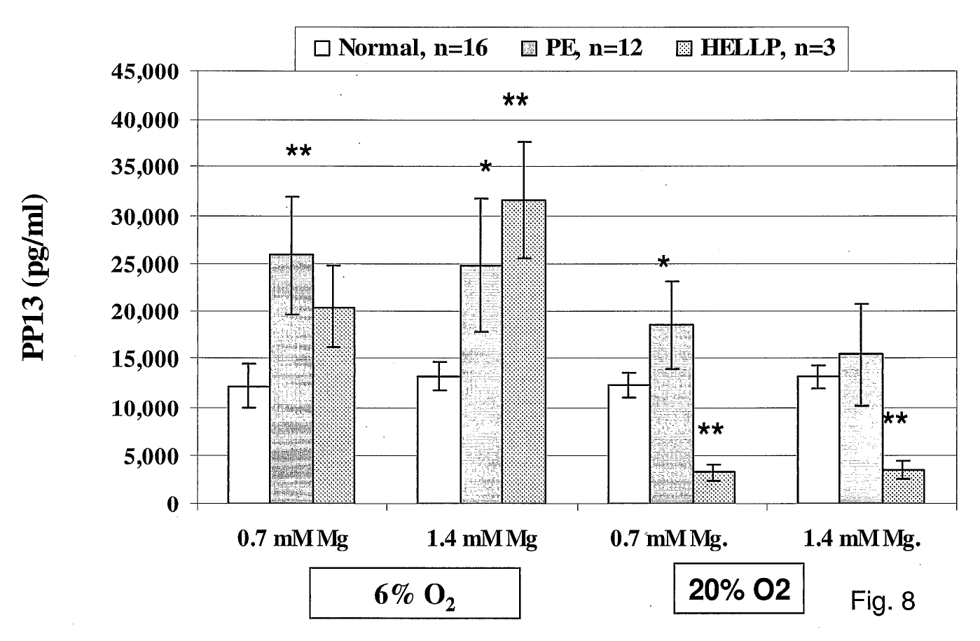

As can be seen in FIG. 7, in cultures grown under an elevated (20%) oxygen level, oxygen had no effect on PP13 release from placental extracts obtained from normal women but decreased significantly the release of PP13 from placental extracts obtained from preeclampsia women, and even more so in placental extracts obtained from HELLP cases, indicating that the oxygen was harming the placental tissue.

### Example 7

### Assessment of Benefit by Combination of Oxygen and Mg

The explants tested in Example 6 were retested using, in addition to the two oxygen levels, two different levels of magnesium, 0.7 mM and 1.4 mM.

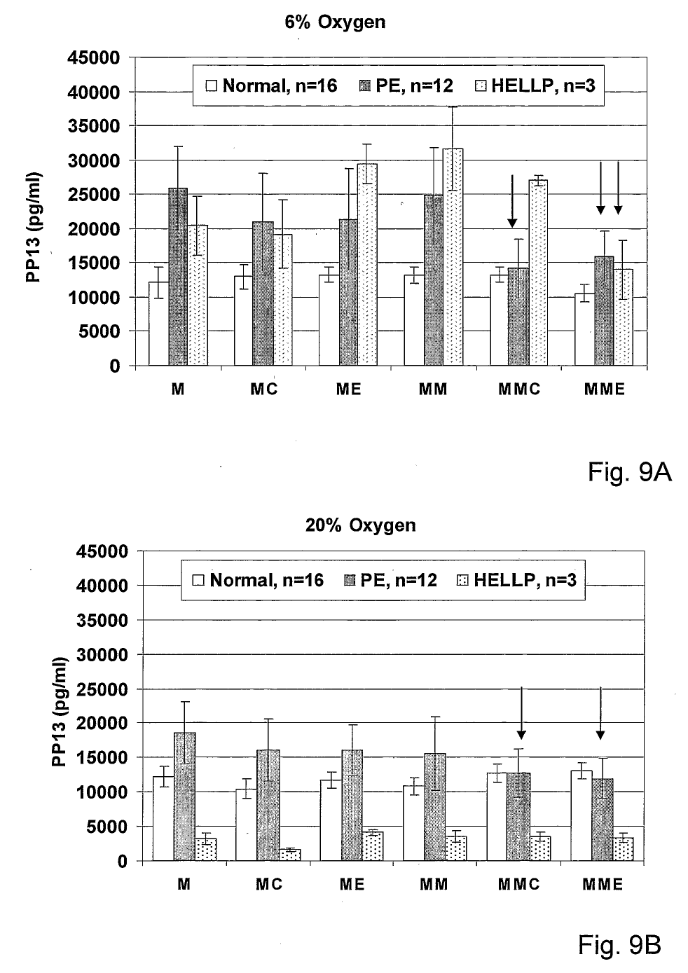

As can be seen in FIG. 8, in the culture grown at elevated oxygen (20%) and MgCl₂ (1.4 mM)—the explants obtained from normal pregnant women were not affected but the preeclamptic explants were brought back to the normal level by magnesium in 20% but not in 6% oxygen. The level of PP13 release in HELLP cases was reduced by 20% O₂ to below the normal level.

### Example 8

### Assessment of Benefit by Combination of Oxygen, Mg and Vitamins C & E

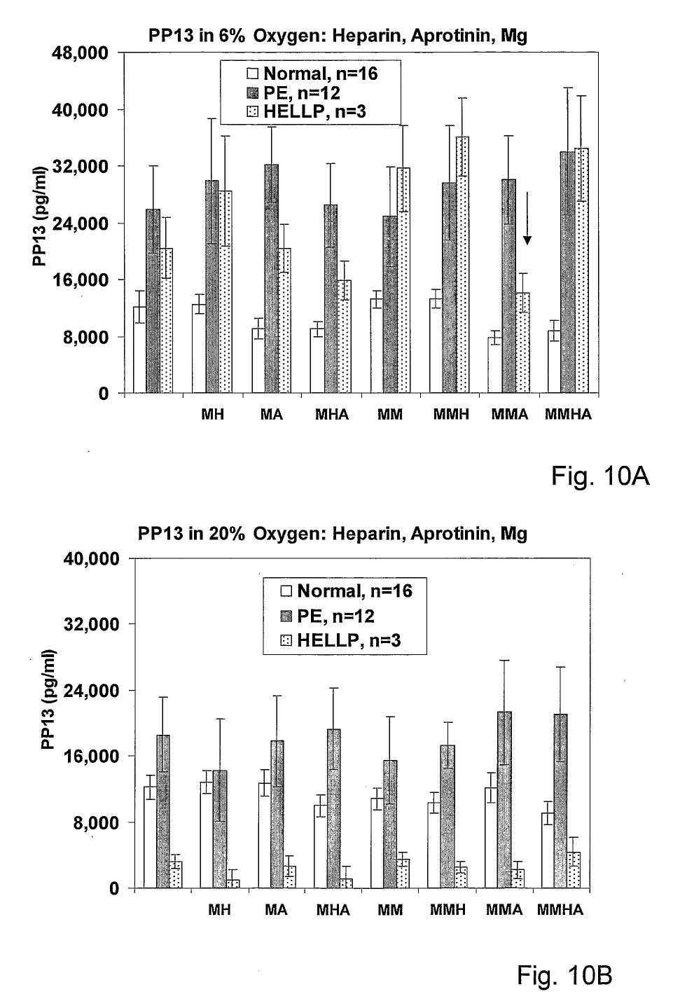

The experiments described in Examples 6 and 7 above were repeated with the addition of Vitamins C & E. As can be seen in FIG. 9, the addition of the vitamins helped to bring PP13 back to the normal level in 6% oxygen, particularly at elevated magnesium. Note that the latter combination also affected the HELLP cases.

### Example 9

### Assessment of Benefit by Combination of Oxygen, Mg and Anticoagulents

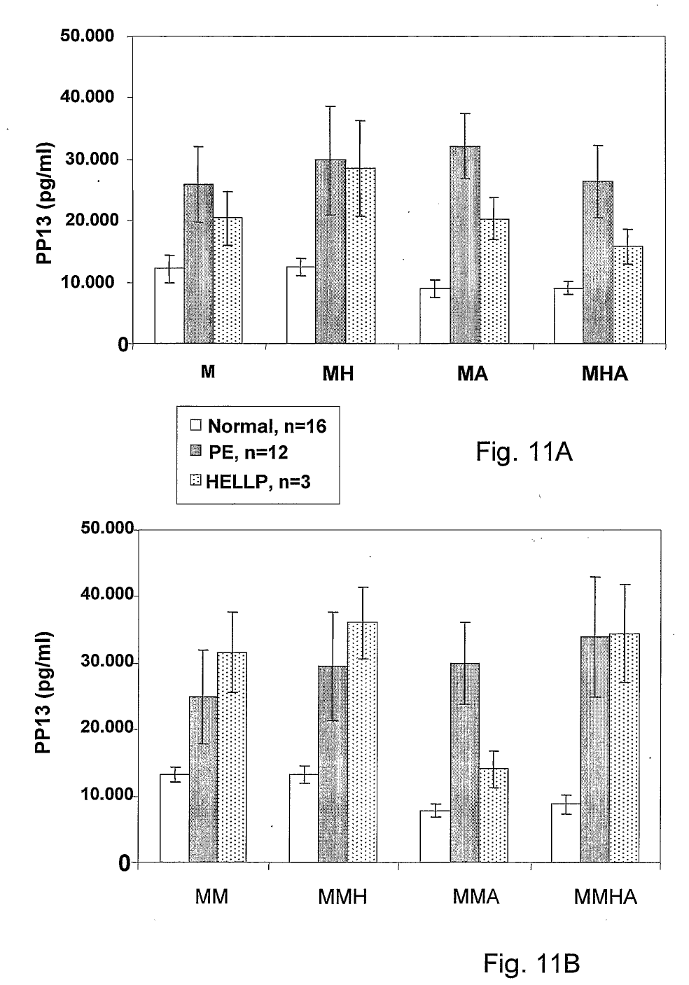

The experiments described in Examples 6 and 7 above were repeated with the addition of various anti-coagulants. As can be seen in FIG. 10, the addition of the anti-coagulants was beneficial particularly if they are combined with magnesium.

### Example 10

### Assessment of Benefit by Combination of Oxygen, Mg and Anti-Coagulants

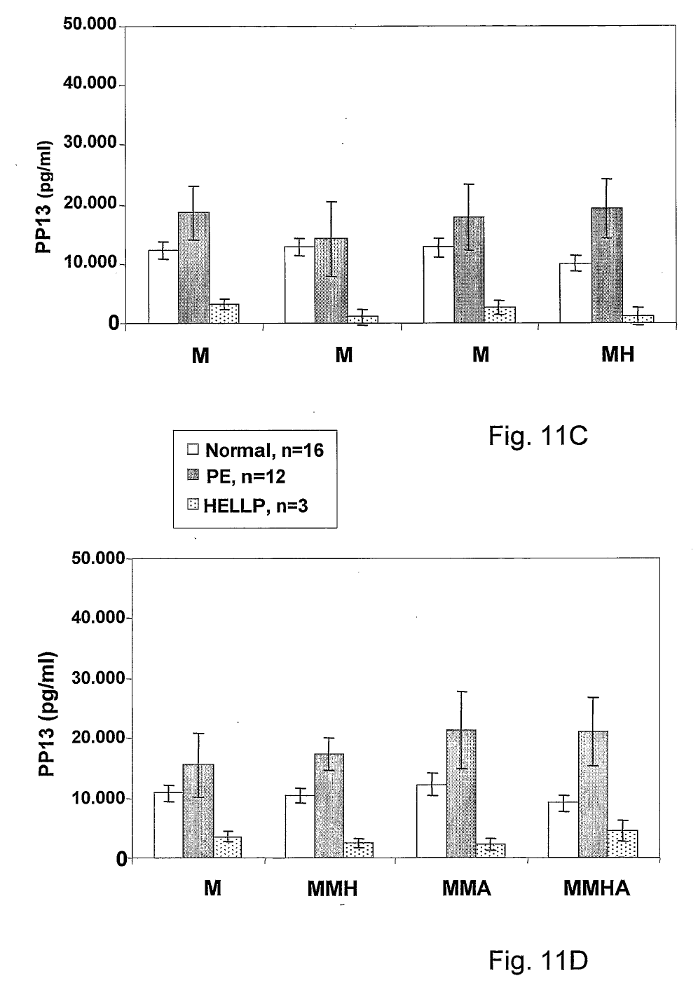

In this example, the effect of heparin and aprotinin on the PP13 release from villous explants of normal, preeclamptic and HELLP placentas was tested in vitro under the conditions of the previous examples. The results are presented in FIGS. 11A-11D.

In FIG. 11A, the explants were cultured under normal conditions (normoxia (6%) and normal Mg (0.7 mM)). Significantly more PP13 was released from explants derived from preeclampsia and HELLP patients than from the normal controls. In FIG. 1B, culture under normoxia and elevated Mg (1.4 mM) resulted in elevated release of PP13 in the HELLP patients. In FIG. 11C (hyperoxia (20%) and normal Mg), significantly less PP13 release occurred from explants of preeclampsia as compared to normoxia, while the control remained at the same level. An almost complete halt in PP13 release from the HELLP patients was noted. An increase in Mg (FIG. 1D) brought about an elevated release of PP13 in the HELLP patients.

With respect to the anti-coagulants, under normoxia and high Mg (FIG. 11B), aprotinin brings PP13 release in explants derived from HELLP patients almost back to normal. Under hyperoxia and normal Mg (FIG. 11C), heparin brings PP13 almost back to normal.

Thus, the method of the invention may be used to forecast which combination of treatments will be the most effective in overcoming the risk for developing preeclampsia.

It is very important to note that in all of the examples described above, a severe subtype of preeclampsia—HELLP—is not affected in the same way as preeclampsia. Thus, the explant system could help in selecting a proper treatment by testing the effect in vitro using the explant system and assessing the proper treatment for the individual woman and disease. Since explants can be stored by cryopreservation, it is also possible to standardize them for further evaluation and tailoring of drug intervention.

### Example 11

### Assessment of Benefit by Administering VEGF

sflt1, a soluble form of the vascular EGF (VEGF) receptor, was found to be at a higher serum level in the third trimester in women who went on to develop preeclampsia 5 weeks later. This molecule competes with the native blood cell receptor for the hormone VEGF. Experimental models have shown that administering VEGF could prevent/reduce the severity of preeclampsia. One way to follow in-vitro the benefit of the treatment is to measure PP13 level and, if decreased back to the normal level, it could serve to assess the benefit of the treatment.

### 4) Longitudinal Monitoring

### Example 12

The following is a prophetic example describing how the method of the invention may be used to follow the risk of preeclampsia of a woman throughout her pregnancy.

The MoM of PP13 level is defined as “1” for a plurality of unaffected (normal) women. If the maternal serum is tested at 10 weeks (1st trimester) and a woman's PP13 level corresponds to 0.11 MoM as defined by a statistical plurality of pregnant women at the respective gestational week, the woman is established as being at high risk for preeclampsia. PP13 MoM=0.45 is the cutoff of 80% specificity and 85% sensitivity. From the model, her likelihood ratio (LR) is found to be 9 times above normal (5%) or at a risk of 45%. If after two weeks of treatment the MoM doesn't cross the cutoff—she remains at high risk.

Then, 4 weeks later (already 2^{nd} trimester, GW=16), she is re-tested for PP13. Her MoM is now 1.24, and the calculated slope between the two points is 7 while the slope cutoff is 3.0. Thus, the woman is defined as being at continued high risk. From the model, her LR is calculated to be 8.4 times above normal (risk=42%). Her average risk is thus 43.5%. Another treatment with anti-oxidants is now evaluated and her MoM returns to be 1.0, indicating her risk has now been reduced. From the model her LR=2 (corresponding to 10% risk).

She is then tested a third time at 30 weeks (3^{rd} trimester) and her MoM is found to be 1.5, whereas the high-risk cutoff of that week is 1.4 MoM. Accordingly, her LR=8.5 times above normal, and her risk is thus again 42.5%. She is now treated again with anti-oxidants and re-tested at 34 weeks. Her MoM is then found to have declined to 1.2 (below cutoff). Her LR=2, risk is 10%. The treatment is now continued and she delivers at term with blood pressure 85/135 and proteinuria 1+ (not considered as preeclampsia).

### Example 13

### Standardization of Explants to Measure the Effect of Various Drugs (Prophetic Example)

A woman was identified to be at risk for preeclampsia by a first trimester marker such as PP13 or PP13 combined with Doppler—how might explants help to tailor a preventive treatment to her?

Scenario 1: Simple Direct Tailoring with a Diversity of Drugs.
We have standardized culture conditions for first trimester explants or explants obtained after delivery.
Explants could be the ones made of patients after delivery or those obtained by Chorionic villi sampling (CVS) at gestational weeks 10-12 or other placenta biopsies as the case may be.
Standardization means defined median viability index, protein content in the explant, total protein released to the medium, PP13 content in the explant and PP13 release from the explant, among others.
Drug effect in-vitro is the effect of drug applied to the culture medium on PP13 release from the placenta explant after 24 hr to 7 days. The effect is compared to the baseline release as measured without the drug.
The Therapeutic Index is thus the in-vitro relative effectiveness of the drug when applied to explants as measured by the difference between PP13 release with (PP13_{D1}) and without (PP13₀) the drug by the equation: (PP13₀−PP13_{D1})/PP13₀, after normalizing to viability, protein content etc as described above, given all other parameters are the same.
It is important to note that in the first trimester (gestational weeks 6-13) PP13 in diseased patient is lower than Normal. Thus the therapeutic index is to return PP13 release is calculated as drugs that elevate PP13 release. In the third trimester (gestational week 26 and above), PP13 is higher in preeclamptic patients than normal. Thus the therapeutic index is decreasing PP13 release back to Normal.
According to the in-vitro therapeutic index one drug or a drug combinations are selected for the in-vivo interventional medication treatment.
Follow up: after selecting the drug to be administrating to the patient, bi-weekly blood testing follow up is recommended to be carried out by measuring the PP13 in the blood and calculating

PP13 slope=(PP13GW2−PP13_{GW1})/(GW₂−GW₁), where GW₁ and GW₂ represent gestational week at the first and second period of PP13 testing, respectively. The result of the formula defined as the slope that was calculated for each individual. This one is compared to the typical median slope for the cases of preeclampsia vs. normal cases. If for the first period (gestational week 6-13) the normal slope is 3.1 and the preeclamptic slope is 10 and after treatment the slope is going down, every test compared to the one before, than it indicates that the treatment is effective. Otherwise—it is recommended to switch to the drug with the second best therapeutic index.

Scenario 2: Tailor to Preeclampsia “Types”
We measured in the body fluid of a woman a very low level of PP13 in the first trimester, indicating that she is at elevated risk for preeclampsia.
We verify the various other features (like low Doppler pulsatility Index or low PAPP-A).
Based on this set—we type the patient to a preeclampsia group A that corresponds to one type of preeclampsia (such as early onset preeclampsia). Another set of patients will have only low PP13 in the first trimester but none of the other and is referred to as Group B.
In-Vitro explants of Type A are subsequently found to be affected by Drug 1 whereas the explants of Group B the Therapeutic Index indicates that only Drug 2 is effective.
Accordingly, once the markers set of a patient is identified to be belonging to group A, Drug 1 will be selected for treating group 1 and vice versa.
Follow up: after selecting the drug to be administrating to the patient, bi-weekly blood testing of PP13 is carried out for the determination of the slope as described above.

Comment—From what we know today on preeclampsia diversity, the approach could at least narrow down significantly the list of suitable therapeutics means to 1-2 candidates for a group. This seems to be most suitable for cases of IVF where many tests are carried out for each woman. Thus a large set of markers could be used to fine-tune the “patient type (group).

Scenario 3: Indirect method
We standardized growth conditions for placenta explants obtained from after delivery from patient A of known outcome.
We find the drug effectiveness on these sets of explants.
After testing many drugs a drug effectiveness scale is developed.
We expose these standardized explants with their scaled drug effectiveness index to the respective drugs in the presence of serum from normal vs. serum from a patient at elevated risk for preeclampsia (Table 1).
We found the most effective drug in the setting of exposure to drugs in the setting of incubation with normal patient serum and the compared effect in the presence of serum from woman identified to eb at elevated risk for preeclampsia.
We choose the drugs that is the less impaired by the patient serum and apply this drug onto the patient in-vivo.
that is the lesser to be impaired by the serum of the affected patient and tailor it to this patient.
This scenario assume that at elevated risk for preeclampsia, the patient body fluids contain various factors such as sflit, estriol, shbg or others that impair/enhance drug effectiveness and presumably even causing preeclampsia.

If so, drugs that appears beneficial to standardized culture explants wouldn't work when applied together with the patient samples. Results of an actual experiment are presented in Table 4 below. It can be seen that the serum from the patient at high risk for preeclampsia caused serious inhibition of many of the types of treatment. This method allows the selection of the treatment most likely to prove effective.

TABLE 4
Blocking impact of patient serum on the Therapeutic Effectiveness of
anti-preeclamptic drugs as assessed in placenta explants by measuring
the release of PP13 to the culture medium

| | Therapeutic Effectiveness | |
|---|---|---|
| | of anti-PP13 medications | |
| | on PP13 release from | |
| | placenta explants when applied | |
| | with serum from normal and | |
| | preeclamptic patient | |
| | Serum | |
| | from a Normal | Serum from a Patient at |
| Treatment | Patient | high risk for preeclampsia |
| Aspirin | 74% | 40% |
| Heparin | 56% | 20% |
| Aspirin + Heparin | 85% | 67% |
| Aspirin + Heparin + O2 | 97% | 85% |
| Vitamin E | 30% | 0% |
| Magnesium | 50% | 20% |
| Mg + Vitamin E | 95% | 80% |

1. A method for determining the effectiveness of a treatment for preeclampsia of a pregnant woman at risk for preeclampsia, comprising:
(a) determining a first concentration of placental protein 13 (PP13) in a bodily substance of the woman obtained prior to the treatment;
(b) determining a second concentration of PP13 in a bodily substance of the woman obtained after initiation of the treatment; and
(c) comparing said first and second concentrations to a corresponding normal level of PP13 and, based on said comparison, determining the effectiveness of the treatment.

2. The method of claim 1, wherein said first concentration of PP13 is selected from the group consisting of:
(a) a predetermined range of median PP13 concentrations for said bodily substance in a plurality of untreated pregnant women at a similar risk for preeclampsia; or
(b) a measured PP13 concentration of the bodily substance of said pregnant woman prior to receiving the treatment.

3. The method of claim 1 wherein said bodily substance is selected from the group consisting of serum, amniotic fluid, urine, saliva, placental tissue and standardized placenta villi.

4. The method of claim 1, wherein the comparison is made between single measurements of said first concentration and said second concentration.

5. The method of claim 1, wherein the comparison is made between a first slope calculated from a plurality of said first concentrations measured at two or more succeeding time points during the pregnancy of the woman and a second slope calculated from a plurality of said second concentrations measured at two or more succeeding time points during the pregnancy of the woman.

6. The method of claim 1 for determining the relative effectiveness of two or more different treatments for preeclampsia, the method comprising:
(a) determining a first concentration of PP13 in a placental tissue explant of the woman obtained prior to the treatment;
(b) contacting the explant with a first treatment;
(c) determining a second concentration of PP13 in the explant after the treatment;
(d) comparing said first and second concentrations to a corresponding normal level of PP13 and, based on said comparison, determining the effectiveness of the first treatment;
(e) repeating steps (a) to (d) with one or more additional treatments; and
(f) comparing the relative effectiveness of the two or more different treatments.

7. The method of claim 6 wherein the second concentration is measured within 1-4 days after the placenta explant is contacted with the treatment.

8. The method of claim 1 wherein in step (d), the effectiveness of the treatment is determined as follows:
(a) if there is no significant difference between the first and second concentrations, the treatment is not effective;
(b) if the difference between the second concentration and the normal level of PP13 is significantly less than the difference between the first concentration and the normal level, the treatment is effective;
(c) if the difference between the second concentration and the normal level of PP13 is significantly greater than the difference between the first concentration and the normal level, the treatment is damaging.

9. The method of claim 8, wherein the plurality of each of the concentrations is compared to a corresponding plurality of normal levels of PP13.

10. The method of claim 8 wherein the plurality of concentrations is determined over a period of 2-3 weeks.

11. The method of claim 1, wherein the comparison is based on the multiple of the medians (MoM), slope and the woman's likelihood ratio (LR) with receiver operating characteristic (ROC) curves used to establish cutoffs for sensitivity and specificity.

12. The method of claim 1, wherein the comparison is based on normal and diseased populations of pregnant women being divided into quartiles, and the concentrations of PP13 being classified in the appropriate quartile.

13. A method for determining the relative effectiveness of two or more different treatments for preeclampsia of a pregnant woman at risk for preeclampsia, comprising:
(a) providing a plurality of placental tissue explants standardized for release of PP13 in response to various preeclampsia treatments;
(b) contacting a bodily substance of the woman with a first placental tissue explant;
(c) contacting said explant with a first treatment and determining the concentration of PP13 of the explant after said first treatment;
(d) repeating steps (b) and (c) with one or more additional explants and treatments; and
(e) determining the difference between the concentrations of PP13 after said treatments and a corresponding normal level of PP13, the treatment resulting in the smallest difference being the most effective.

14. The method of claim 13 wherein the standardized placenta explants are cryo-preserved.

15. A diagnostic kit for carrying out the method of claim 13 comprising:
(a) a set of anti-preeclampsia drugs; and
(b) a set of standardized placenta explants.

16. A kit according to claim 15 further comprising computer software providing a calculation model to determine the effectiveness of the drugs based on the measured PP13 values.
